(19) United States
(12) Patent Application Publication
(10) Pub. No.: US 2023/0230678 A1
(43) Pub. Date: Jul. 20, 2023

(54) AD HOC MODEL BUILDING AND MACHINE LEARNING SERVICES FOR RADIOLOGY QUALITY DASHBOARD

(71) Applicant: KONINKLIJKE PHILIPS N.V., EINDHOVEN (NL)

(72) Inventors: Prescott Peter KLASSEN, CAMBRIDGE, MA (US); Thomas BUELOW, GROSSHANSDORF (DE); Tim Philipp HARDER, AHRENSBURG (DE)

(21) Appl. No.: 17/928,945

(22) PCT Filed: Jun. 4, 2021
(86) PCT No.: PCT/EP2021/064961

§ 371 Date: Dec. 1, 2022

Related U.S. Application Data
(60) Provisional application No. 63/035,033, filed on Jun. 5, 2020.

(51) Int. Cl. G16H 30/40; G16H 15/00

(52) U.S. Cl. CPC G16H 30/40; G16H 15/00

(57) ABSTRACT

A method (100) of generating and using one or more radiology analysis tools comprising: providing a labeling user interface (28, 40) on the workstation via which a user creates a labeled dataset by defining label types; receiving a user selection of desired output as at least one of the defined label types; identifying a proposed machine learning (ML) model based on the defined label types and the desired output; providing one or more GUI dialogs (40) presenting the proposed ML model and allowing the user to generate a user-designed proposed ML model (38) from the proposed ML model; training the user-designed ML model using training data comprising at least a portion of the labeled dataset, thereby generating a trained ML model (44); and deploying the trained ML model for an analysis process applied to at least a portion of radiology images and/or radiology reports in.

### FIELD

The following relates generally to the radiology arts, radiology reading arts, radiology department performance assessment arts, radiology report quality assessment arts, machine-learning arts, and related arts.

### BACKGROUND

Radiology departments at hospitals or other large medical institutions perform a large number of imaging examinations targeting different anatomies (e.g. head, lungs, limbs, et cetera) for different purposes (oncology, cardiology, pulmonology, bone fracture assessment, et cetera), and using different imaging devices often of different imaging modalities such as magnetic resonance imaging (MRI), computed tomography (CT), positron emission tomography (PET), X-ray, ultrasound, et cetera. A radiology department employs a staff of imaging technicians who operate the imaging devices, and a staff of radiologists who read the imaging examinations. In a large radiology department, there may be multiple work shifts so that the imaging devices are miming over long time intervals (up to 24 hours per day in some cases), and likewise there may be multiple radiologist work shifts to read the large number of generated imaging examinations.

Assessing the work product of such a complex radiology department is difficult. Typically, radiology reports retrieved from Radiology Information Systems (RIS) and images retrieved from Picture Archiving and Communication Systems (PACS) are analyzed, and feedback is provided on the qualities of the reports and the images. The feedback is generated using algorithms to specifically address particular measures of quality retrospectively against archived reports and images. Users are able to set custom parameters and date ranges in order to select specific subsets of images and reports and create unique collections to report on.

The users are likely to be radiology department managers or the like who may have medical/radiology expertise, but are unlikely to have a background in in computer science, machine learning, or the like. In addition, in typical systems, ML algorithms and modeling platforms are typically standalone products that require users to learn how to work within specific environments in order to run experiments and evaluate performance.

The following discloses certain improvements to overcome these problems and others.

### SUMMARY

In one aspect, a non-transitory computer readable medium stores instructions readable and executable by at least one electronic processor to provide statistical analysis on one or more radiology databases in conjunction with a workstation having a display device and one or more user input devices. The instructions comprising: instructions readable and executable by the at least one electronic processor to define a library of model components; labeling user interface (UI) instructions readable and executable by the at least one electronic processor to provide a labeling UI on the workstation via which a user creates a labeled dataset of labeled radiology images and/or labeled radiology reports by defining label types and adding labels of the defined label types to user-selected radiology images and/or radiology reports in the one or more radiology databases; model building instructions readable and executable by the at least one electronic processor provide a model building UI on the workstation via which the user selects a desired output as at least one of the defined label types and selects and interconnects model components of the library of model components to construct a proposed ML model outputting the desired output; model training instructions readable and executable by the at least one electronic processor to train the user-designed ML model using training data comprising at least a portion of the labeled dataset thereby generating a trained ML model; and analysis instructions readable and executable by the at least one electronic processor to perform an analysis task on at least a portion of the radiology images and/or radiology reports in the one or more radiology databases using the trained ML model and present results of the analysis task on an analysis UI on the workstation.

In another aspect, a method of generating and using one or more radiology analysis tools performed in conjunction with a workstation having a display device and one or more user input devices includes: providing a labeling UI on the workstation via which a user creates a labeled dataset of labeled radiology images and/or labeled radiology reports by defining label types and adding labels of the defined label types to user-selected radiology images and/or radiology reports in one or more radiology databases; receiving, via the workstation, a user selection of a desired output as at least one of the defined label types; identifying a proposed ML model based on the defined label types and the desired output; providing, on the GUI, one or more GUI dialogs presenting the proposed ML model and allowing the user to generate a user-designed proposed ML model from the proposed ML model; training the user-designed ML model using training data comprising at least a portion of the labeled dataset, thereby generating a trained ML model; and deploying the trained ML model for an analysis process applied to at least a portion of the radiology images and/or radiology reports in the one or more radiology databases.

In another aspect, an apparatus for generating and using one or more radiology analysis tools includes a display device and one or more user input devices. At least one electronic processor is programmed to: provide a labeling UI on the display device via which a user creates a labeled dataset of labeled radiology images and/or labeled radiology reports by defining label types and adding labels of the defined label types to user-selected radiology images and/or radiology reports in one or more radiology databases; receive a user selection of a desired output as at least one of the defined label types; identify a proposed ML model based on the defined label types and the desired output; provide, on the labeling UI, one or more GUI dialogs presenting the proposed ML model and allowing the user to generate a proposed ML model from the proposed ML model; train the user-designed ML model using training data comprising at least a portion of the labeled dataset, thereby generating a trained ML model; evaluate the trained ML model on evaluation data comprising a portion of the radiology images and/or radiology reports in the one or more radiology databases; and deploy the trained ML model for an analysis process applied to at least a portion of the radiology images and/or radiology reports in the one or more radiology databases.

One advantage resides in providing an apparatus suggesting ML models for a user analyzing a radiology database.

Another advantage resides in providing an apparatus for a user to generate a ML tool to guide the user through a series of analysis workflow steps.

Another advantage resides in providing an apparatus for a user to provide the user with interactive decision points where users can provide additional filters or parameters to inform the next step of the workflow.

Another advantage resides in enabling a radiologist or other radiology department analyst to apply their own knowledge to an analysis of a radiology report.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for analyzing radiology reports in accordance with the present disclosure.

FIG. 2 shows exemplary flow chart operations performed by the apparatus of FIG. 1.

FIG. 3 shows an illustrative example of a labeling user interface implemented on the apparatus of FIG. 1.

FIG. 4 shows an illustrative example of a dashboard provided by a user interface implemented on the apparatus of FIG. 1.

### DETAILED DESCRIPTION

This following relates to an apparatus providing a dashboard or other graphical user interface (GUI) that allows a radiology department quality manager or other analyst to perform various analyses on images stored in a Picture Archiving and Communication System (PACS), possibly in relation to other information such as patient demographic information from a Radiology Information System (RIS) database or radiology findings stored in the PACS.

The GUI may be designed to implement certain predefined analyses using built-in algorithmic or machine-learning (ML) tools. However, it is anticipated that users may want to perform other analyses not provided in the dashboard. These may be analyses specific to the particular hospital or medical institution, e.g. comparing image characteristics for images generated by a newly added magnetic resonance imaging (MRI) scanner with those generated by the existing MRI scanners. Similarly, it might be desired to compare images generated by a specific technologist with images acquired by other technologists. There are numerous other reasons an analysis other than those provided by the dashboard might be desired, such as analyses based on new radiology science developments or so forth.

Users of such a dashboard are likely to be radiology department managers, clinical directors, operations managers, or the like who may have medical/radiology expertise, but are unlikely to have a background in computer science, machine learning, or the like.

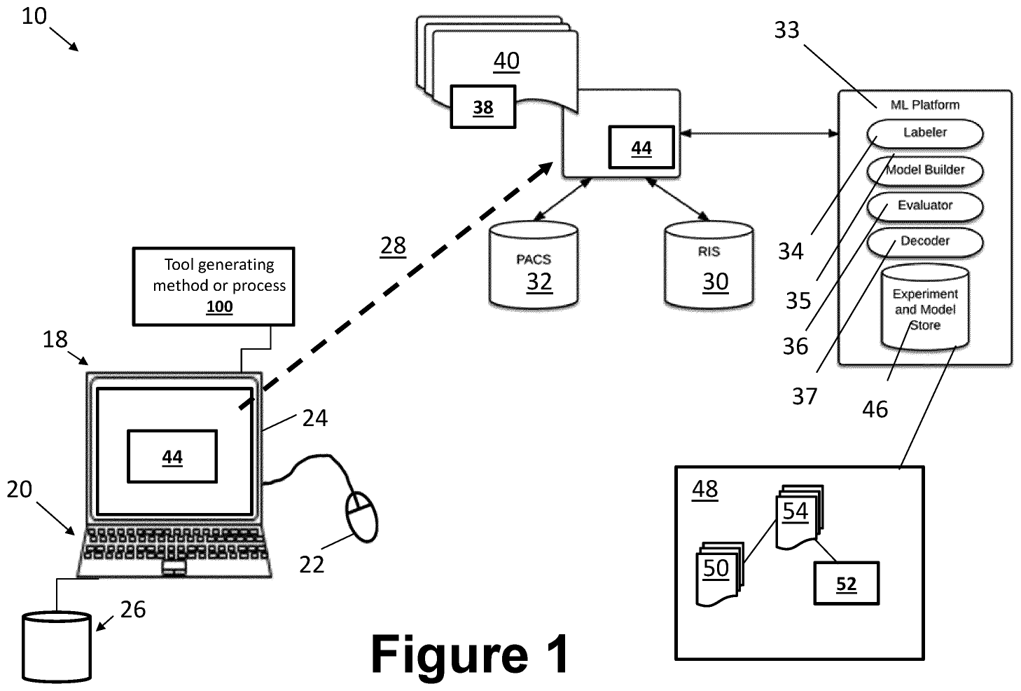

To service such users, the disclosed systems and methods enable such a user to develop and deploy new ML based tools. As diagrammatically shown in FIG. 1, an illustrative system includes a data labeler 34, a model builder 35, a model evaluator 36, and a decoder 37. These components have associated user interface (UI) menus and views for user interaction in a non-technical way (that is, non-technical from a computer science standpoint).

The labeler 34 allows the user to create an annotated training data set according to a user-defined labeling schema. For example, if the user desires to develop a ML tool for assessing whether skin folds in a mammography image tend to lead to a lower incidence of a lesion being found (whether correctly or incorrectly), then the images may be labeled with a label indicating presence/absence of a skin fold, and may be labeled with the clinical finding from the corresponding radiology report.

The model builder 35 then generates the ML model. Based on the data types of the labels and optionally other user-supplied input, along with an indication of the desired output of the ML component, the model builder proposes to the user an ML model for use in building the tool. The system is preferably modular insofar as additional ML models can be added, with suitable application programming interfaces (APIs) for connecting with the system. The model builder phase may also define the inputs to the model and the desired output, e.g. the label to be predicted or, more generally, the output. For ML applied to images, the ML model is contemplated to include a CNN or other type of ML component that receives images as a whole (i.e., without breaking the images down into features characterizing image patches or the like), although ML models that employ (optionally user-defined) image patches are also contemplated. For text-based inputs, by way of nonlimiting illustrative example various natural language processing (NLP) components, and/or an SVM classifier, may be proposed by the system.

In a training phase, the model builder 35 then trains the chosen model using (at least a portion of) the labeled data generated by the user via the labeler. This phase uses a standard training algorithm suitable for the chosen ML model. For example, an SVM has as adjustable parameters the hyperplane parameters w and b of the hyperplane given in standard form by wx+b=0 (where x is a vector of input values and w is a (not necessarily normalized) normal vector to the hyperplane. Hence, a suitable training algorithm for an SVM may employ iterative optimization of b and the components of w. In the case of a ML component employing a CNN or other artificial neural network (ANN), the adjustable parameters include, for example, weights, SoftMax or other activation function parameters, or other parameters of the propagation functions connecting the neural network layers, and a technique such as backpropagation can be employed to optimize the ANN parameters. These are merely illustrative examples. The output of the model trainer is the trained ML model.

The model evaluator 36 then evaluates the trained ML model. In one approach, a portion of the labeled data is not used in training, and is instead used as test data or validation data for the model evaluation. In some cross-validation approaches, the model training and model validation phases may be performed iteratively, e.g. in k-fold cross-validation the labeled dataset is randomly partitioned into k sub-sets (or “folds”), where k−1 of these folds are used for training followed by validation using the remaining fold. This process can be repeated with a different fold randomly designated as the validation fold in each iteration. In another evaluation approach, all labeled data is used in the training, and the system then chooses suitable unlabeled data from the PACS, RIS, or other database to analyze and present the results to the user, who manually assesses the model performance.

The decoder 37 implements the deployment phase, in which the trained and evaluated (e.g. validated) ML model is deployed for an analytical task whose results are intended to be used in assessing some aspect of radiology department performance. In the decoder phase, the trained and evaluated ML model is applied to data other than (or additional to) the data of the labeled dataset.

With continuing reference to FIG. 1, an illustrative apparatus 10 for performing a statistical analysis on radiological data is shown. The apparatus 10 includes an electronic processing device 18, such as a workstation computer, or more generally a computer. The workstation 18 may also include a server computer or a plurality of server computers, e.g. interconnected to form a server cluster, cloud computing resource, or so forth, to perform more complex image processing or other complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The workstation 18 is also in communication with one or more radiology databases, such as a RIS 30 and a PACS 32 (among others, such as an EMR, EHR and so forth). The workstation 18 is configured to retrieve information about the radiology examination (e.g., from the RIS), and/or images acquired during the examination (e.g., from the PACS) to perform a statistical analysis of the data stored in the RIS 30 and the PACS 32. Optionally, the workstation 18 is further configured to retrieve patient data.

The non-transitory computer readable medium 26 is configured to store instructions are readable and executable by the at least one electronic processor 20 of the workstation 18 to perform disclosed operations to analyze the data from the RIS 30 and the PACS 32. To do so, the non-transitory computer readable medium 26 can include a ML platform 33 that includes one or more modules, such as the illustrative labeler module 34, model builder 35, model evaluator 36, and model decoder 37, executable by the at least one electronic processor 20. The modules 34, 35, 36, 37 are configured to generate one or more proposed ML models 38 made up of one or more ML components 39 with various user-defined configurations. Moreover, the non-transitory computer readable medium 26 is configured to store a plurality of GUI dialogs 40 (e.g., a pop-up window, or a window comprising the entire screen of the display device 24) for display on the display device 24 via the GUI 28. The GUI dialogs 40 can, for example, include GUI dialogs for: interfacing with the labeler model 34 via which a user can bring up images or radiology reports and label them in various ways (e.g., image has/does not have a skin fold; report does/does not meet a certain quality criterion, et cetera) so as to create the labeled training dataset; proposing a ML model to the user and for enabling the user to define the inputs to the model and the desired model output; presenting evaluation results to the user (in automated validation embodiments) or present images or reports used in validation along with the ML-generated output for confirmation or correction by the user (in manual evaluation embodiments); and display visualizations of the proposed ML model 38 applied by the decoder 37. The non-transitory computer readable medium 26 also includes an experiment and model storage 46 configured to store trained ML models 44 generated by the user.

In some embodiments, the instructions include instructions to define a library 48 of model components, which can be stored in the experiment and model storage 46. The library 48 of model components can includes a plurality of ML components 50, such as, for example, at least one artificial neural network (ANN) component, at least one support vector machine (SVM) component, and at least one statistical analysis component, among others. At least one feature extraction component 52 is configured to extract (i) image features from radiology images in the PACS 32, and label the radiology images with the extracted image features, and/or (ii) report features from radiology reports stored in the RIS 30, stored and label the radiology reports with the extracted report features. Moreover, the library 48 can include one or more application programming interfaces (APIs) for the ML components 50 and the feature extraction component.

In some embodiments, the instructions can include instructions to provide one or more of the GUI dialogs 40 on the workstation. For example, as shown in FIG. 1, the ML platform 33 includes a labeler module 34 configured to create, store, and maintain label schemas and labeling instances. To do so, the labeler module 34 is configured to analyze data from the RIS 30 and/or the PACS 32, and generate the label schemas therefrom. The labeler module 36 is configured to store controlled vocabularies for classification, as well as taxonomies and ontologies that may be used to label reports or images. The labeling may be applied to content at different levels of granularity: cohort, report, sentence, phrase, token. A user can use the GUI labelling dialog 40 to create a labeled dataset of labeled radiology images and/or labeled radiology reports by defining label types and adding labels of the defined label types to user-selected radiology images in the PACS 32 and/or radiology reports in the RIS 30.

In some embodiments, the ML platform 33 includes the model builder module 34. In some examples, the model builder module 34 is configured to implement model building instructions to provide a model building UI 40 on the workstation 18 via which the user selects a desired output as at least one of the defined label types and selects and interconnects model components of the library 48 of model components to construct a user-designed ML model outputting the desired output. In addition, the model builder module 34 is configured to implement model training instructions to train the user-designed ML model using training data comprising at least a portion of the labeled dataset thereby generating a trained ML model 44.

In some embodiments, the ML platform 33 includes the evaluator module 35 configured to implement model evaluation to perform an evaluation of the trained ML model 44 by applying the trained ML model to evaluation data comprising a portion of the radiology images and/or radiology reports in the one or more radiology databases. An evaluation UI 40 is provided to present evaluation results summarizing the output of the trained ML model 44 applied to the evaluation data.

In some embodiments, the instructions can include model storage and retrieval instructions for storing the trained ML models 44 in the experiment and model storage 46 of the non-transitory computer readable medium 26. The model storage and retrieval instructions also include instructions to retrieve a trained ML model 44 and invoke analysis instructions to perform an analysis task using the retrieved ML model.

In some embodiments, the ML platform 33 includes a decoder module 37 configured to implement analysis instructions to perform an analysis task on at least a portion of the radiology images in the PACS 32 and/or radiology reports in the RIS 30 using the trained ML model 44. Results of the analysis task can be presented on an analysis UI 40 on the workstation 18.

The apparatus 10 is configured as described above to perform a method or process 100 for generating and using one or more radiology analysis tools. The non-transitory storage medium 26 stores instructions which are readable and executable by the at least one electronic processor 20 of the workstation 18 to perform disclosed operations including performing the method or process 100 for generating and using one or more radiology analysis tools. In some examples, the method 100 may be performed at least in part by cloud processing.

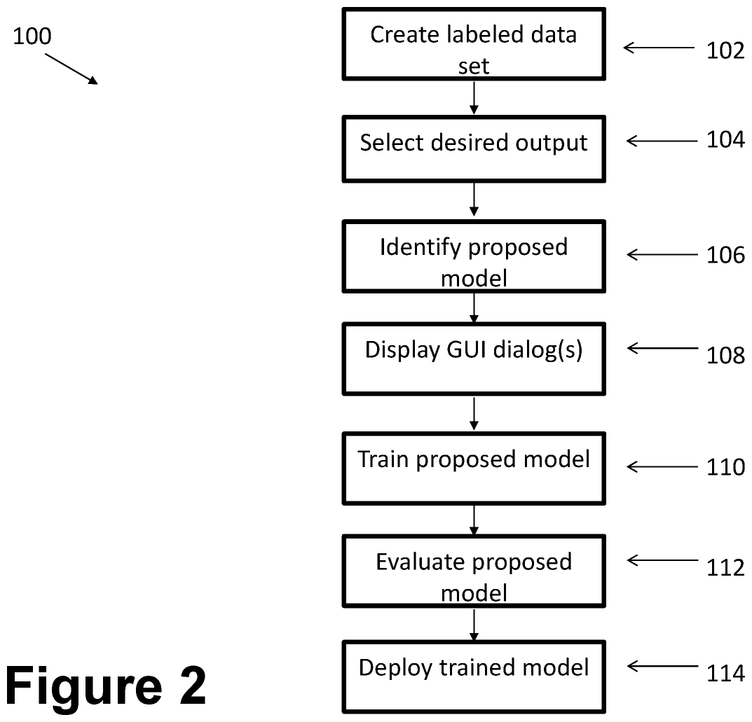

With reference to FIG. 2, and with continuing reference to FIG. 1, an illustrative embodiment of the method 100 is diagrammatically shown as a flowchart. At an operation 102, a labeling GUI 28 on the workstation 18 via which a user creates a labeled dataset of labeled radiology images in the PACS 32 and/or labeled radiology reports in the RIS 30 by defining label types and adding labels of the defined label types to user-selected radiology images and/or radiology reports. The user can use the at least one user input device 22 to create the labeled dataset.

At an operation 104, a user selection (via the at least one user input device 22) of a desired output as at least one of the defined label types is received at the workstation 18.

At an operation 106, a proposed ML model 38 identified based on the defined label types (at the operation 102) and the desired output (at the operation 104). For example, the proposed ML model 38 comprises an artificial neural network (ANN), at least one support vector machine (SVM), or a statistical analysis. In some embodiments, the proposed ML model 38 is configured to receive images from the PACS 32. In other embodiments, the proposed ML model 38 is configured to receive features extracted from images (e.g., from the feature extraction component 52). In further embodiments, the proposed ML model 38 is configured to receive features extracted from radiology reports (e.g., from the feature extraction component 52) using natural language processing (NLP) operations.

At an operation 108, the one or more GUI dialogs 40 are provided on the GUI 28 to present the proposed ML model 38. Using the GUI dialogs 40, the user can generate a user-designed model from the proposed ML model 38.

At an operation 110, the user-designed ML model is trained using training data comprising at least a portion of the labeled dataset to generate generating a trained ML model 44.

At an operation 112, the trained model 44 is evaluated on evaluation data comprising a portion of the radiology images in the PACS 32 and/or radiology reports in the RIS 30. In some examples, the evaluating includes evaluating the trained ML model 44 on data other than the labeled dataset. In other examples, the evaluating includes evaluating the trained ML model 44 by comparing outputs of the trained ML model applied to images and/or radiology reports of the labeled dataset with corresponding labels of the images and/or radiology reports of the labeled dataset.

At an operation 114, the trained ML model 44 is deployed for an analysis process applied to at least a portion of the radiology images in the PACS 32 and/or radiology reports in the RIS 30.

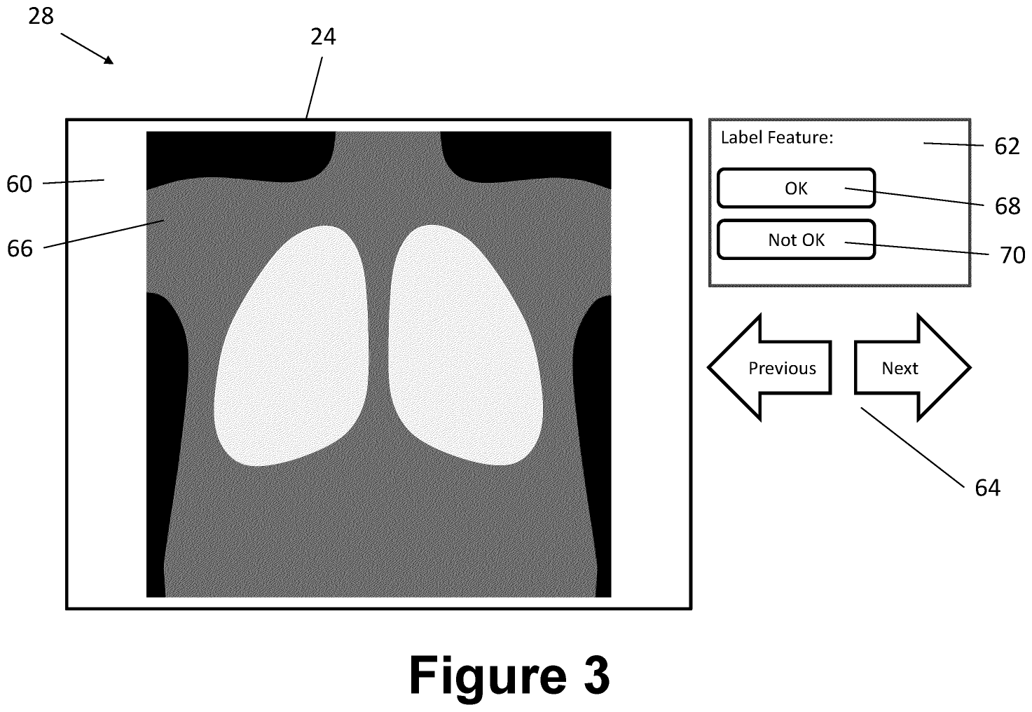

With reference to FIG. 3, an illustrative example of the labeling UI 28 is shown. The labelling UI 28 comprises an image or report viewing area 60, a label area 62 and a navigation area 64. As shown in FIG. 3, the image viewing area 60 displays a chest X-ray image 66 for which a label is to be assigned. To assign a label to the image 66, a user can use an “OK” button 68 or a “Not OK” button 70 (or variants thereof) indicating whether the particular label is acceptable. In order to annotate a larger amount of image necessary for training, the user can navigate cases using the buttons in the navigation area 64 to proceed to the next case or revisit the previous case.

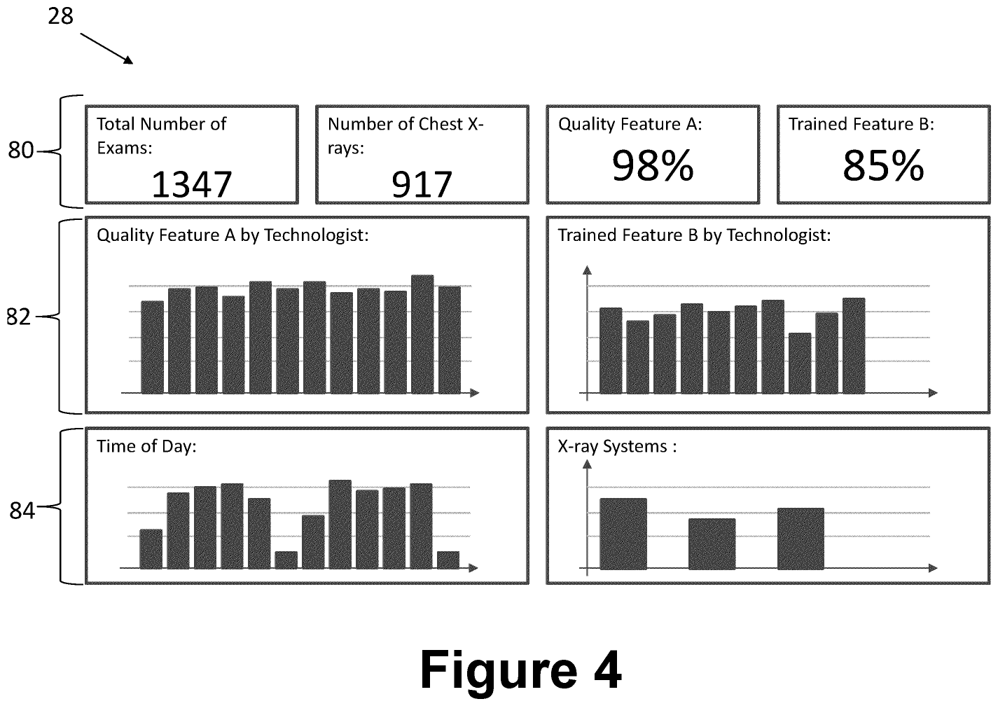

With reference to FIG. 4, an illustrative example of a dashboard provided by the GUI 28 of data related to the trained ML model 44 is shown. As shown in FIG. 4, a set of quantitative indicators 80 shows a quick overview over the data in the non-transitory computer readable medium 26 and/or the RIS database 30 and/or the PACS database 32. As shown in FIG. 4, four quantitative indicators 80 are shown, and can include, for example, a total number of reports, a number of chest X-rays, a percentage of cases for which a quality feature “A” was assessed to be present, and a percentage of cases for which a newly trained classifier “B” was assessed to be acceptable. The dashboard also includes one or more plots 82 for data per technologist. For example, FIG. 4 shows a first plot 82 as a bar graph showing percentages of the quality feature A per technologist, and a second bar graph showing percentages of the newly trained classifier B per technologist. In addition, the dashboard also shows one or more plots 84 of overall performance statistics. For example, FIG. 4 shows a plot 84 as a bar graph showing a number of case per hour of the day, and a second plot as a bar graph showing a number of cases per x-ray system. These are merely examples, and should not be construed as limiting.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions readable and executable by at least one electronic processor to provide statistical analysis on one or more radiology databases in conjunction with a workstation having a display device and one or more user input devices, the instructions comprising:
instructions readable and executable by the at least one electronic processor to define a library of model components;
labeling user interface (UI) instructions readable and executable by the at least one electronic processor to provide a labeling UI on the workstation via which a user creates a labeled dataset of labeled radiology images and/or labeled radiology reports by defining label types and adding labels of the defined label types to user-selected radiology images and/or radiology reports in the one or more radiology databases;
model building instructions readable and executable by the at least one electronic processor provide a model building UI on the workstation via which the user selects a desired output as at least one of the defined label types and selects and interconnects model components of the library of model components to construct a proposed ML model outputting the desired output;
model training instructions readable and executable by the at least one electronic processor to train the user-designed ML model using training data comprising at least a portion of the labeled dataset thereby generating a trained ML model; and
analysis instructions readable and executable by the at least one electronic processor to perform an analysis task on at least a portion of the radiology images and/or radiology reports in the one or more radiology databases using the trained ML model and present results of the analysis task on an analysis UI on the workstation.

2. The non-transitory computer readable medium of claim 1, wherein the library of model components includes at least:
a plurality of machine learning (ML) components;
at least one feature extraction component configured to extract image features from radiology images and label the radiology images with the extracted image features, and/or configured to extract report features from radiology reports and label the radiology reports with the extracted report features, and
application programming interfaces (APIs) for the ML components and the at least one feature extraction component.

3. The non-transitory computer readable medium of claim 2, wherein the plurality of ML components includes at least one artificial neural network (ANN) component, at least one support vector machine (SVM) component, and at least one statistical analysis component.

4. The non-transitory computer readable medium of claim 1, wherein the instructions further comprise:
model evaluation instructions readable and executable by the at least one electronic processor to perform an evaluation of the trained ML model by applying the trained ML model to evaluation data comprising a portion of the radiology images and/or radiology reports in the one or more radiology databases and providing an evaluation UI presenting evaluation results summarizing the output of the trained ML model applied to the evaluation data.

5. The non-transitory computer readable medium of claim 1, wherein the instructions further comprise:
model storage and retrieval instructions readable and executable by the at least one electronic processor to:
store trained ML models on the non-transitory computer readable medium, and
retrieve a trained ML model from the non-transitory storage medium and invoke the analysis instructions to perform an analysis task using the retrieved ML model.

6. A method of generating and using one or more radiology analysis tools performed in conjunction with a workstation having a display device and one or more user input devices, the method comprising:
providing a labeling UI on the workstation via which a user creates a labeled dataset of labeled radiology images and/or labeled radiology reports by defining label types and adding labels of the defined label types to user-selected radiology images and/or radiology reports in one or more radiology databases;
receiving, via the workstation, a user selection of a desired output as at least one of the defined label types;
identifying a proposed machine learning model based on the defined label types and the desired output;
providing, on the GUI, one or more GUI dialogs presenting the proposed ML model and allowing the user to generate a user-designed proposed ML model from the proposed ML model;
training the user-designed ML model using training data comprising at least a portion of the labeled dataset, thereby generating a trained ML model; and
deploying the trained ML model for an analysis process applied to at least a portion of the radiology images and/or radiology reports in the one or more radiology databases.

7. The method of claim 6, further comprising, prior to the deploying:
evaluating the trained ML model on evaluation data comprising a portion of the radiology images and/or radiology reports in the one or more radiology databases.

8. The method of claim 7, wherein the proposed ML model comprises an artificial neural network (ANN), at least one support vector machine (SVM), or a statistical analysis.

9. The method of claim 6, wherein the proposed ML model is configured to receive images.

10. The method of claim 6, wherein the proposed ML model is configured to receive features extracted from images.

11. The method of claim 6, wherein the proposed ML model is configured to receive features extracted from radiology reports using natural language processing (NLP).

12. The method of claim 7, wherein: the evaluating includes evaluating the trained ML model on data other than the labeled dataset.

13. The method of claim 7, wherein: the evaluating includes evaluating the trained ML model by comparing outputs of the trained ML model applied to images and/or radiology reports of the labeled dataset with corresponding labels of the images and/or radiology reports of the labeled dataset.

14. An apparatus for generating and using one or more radiology analysis tools, the apparatus comprising:
a display device;
one or more user input devices; and
at least one electronic processor programmed to:
provide a labeling user interface (UI) on the display device via which a user creates a labeled dataset of labeled radiology images and/or labeled radiology reports by defining label types and adding labels of the defined label types to user-selected radiology images and/or radiology reports in one or more radiology databases;
receive a user selection of a desired output as at least one of the defined label types;
identify a proposed machine learning (ML) model based on the defined label types and the desired output;
provide, on the labeling UI, one or more GUI dialogs presenting the proposed ML model and allowing the user to generate a proposed ML model from the proposed ML model;
train the user-designed ML model using training data comprising at least a portion of the labeled dataset, thereby generating a trained ML model;
evaluate the trained ML model on evaluation data comprising a portion of the radiology images and/or radiology reports in the one or more radiology databases; and
deploy the trained ML model for an analysis process applied to at least a portion of the radiology images and/or radiology reports in the one or more radiology databases.

15. The apparatus of claim 14, wherein the proposed ML model comprises an artificial neural network (ANN), at least one support vector machine (SVM), or a statistical analysis.

16. The apparatus of claim 14, wherein the proposed ML model is configured to receive images.

17. The apparatus of claim 14, wherein the proposed ML model is configured to receive features extracted from images.

18. The apparatus of claim 14, wherein the proposed ML model is configured to receive features extracted from radiology reports using natural language processing (NLP).

19. The apparatus of claim 14, wherein the at least one electronic processor is programmed to:
evaluate the trained ML model on data other than the labeled dataset.

20. The apparatus of claim 14, wherein the at least one electronic processor is programmed to:
evaluate the trained ML model by comparing outputs of the trained ML model applied to images and/or radiology reports of the labeled dataset with corresponding labels of the images and/or radiology reports of the labeled dataset.
